(19) United States
(12) Patent Application Publication
(10) Pub. No.: US 2009/0074735 A1
(43) Pub. Date: Mar. 19, 2009

(54) TOPICAL PRODUCT CONTAINING SACRIFICIAL MATERIAL FOR NEUTRALIZING FREE RADICALS

(76) Inventor: Ashok V. Joshi, Salt Lake City, UT (US)

Correspondence Address: CERAMATEC, INC., 2425 SOUTH 900 WEST, SALT LAKE CITY, UT, 84119, US

(21) Appl. No.: 12/234,409

(22) Filed: Sep. 19, 2008

Related U.S. Application Data
(60) Provisional application No. 60/973,515, filed on Sep. 19, 2007.

(51) Int. Cl. A61K 33/24; A61K 33/26; A61K 33/06; A61P 17/18; A61K 35/66; A61K 35/74; A61K 33/30; A61K 33/34

(52) U.S. Cl. 424/93.45; 424/646; 424/682; 424/642; 424/630; 424/653; 424/617; 424/650; 424/93.48; 424/93.51

(57) ABSTRACT

A topical product to protect the skin from free radicals is disclosed in one embodiment of the invention as including a carrier medium for topical application to the skin. A sacrificial material that is compatible with the skin is interspersed with the carrier medium. This sacrificial material may be selected to react with free radicals within the skin. The sacrificial material may be further selected to react with water to produce hydrogen (a powerful antioxidant) and a reaction product that is also compatible with the skin. In this way, both the sacrificial material and the hydrogen will react with free radicals to provide protection against free radicals.

### RELATED APPLICATIONS

This application claims priority to U.S. Provisional Patent No. 60/973,515 filed on Sep. 19, 2007 and entitled Sacrificial Anode-Infused Topical Product.

### BACKGROUND OF THE INVENTION

1. Field of the Invention

This invention relates to sunscreens and cancer prevention, and more particularly to products and methods for reducing the effects of free radicals on, within, or under the skin.

2. Description of the Related Art

It is a generally accepted fact that sunlight has a significant and profound effect on human skin. Sunlight typically contains three types of ultraviolet radiation, each having different wavelengths and different effects on human skin: UVA, UVB, and UVC radiation. UVC radiation is mostly absorbed by ozone layers, and thus its effect on the skin is minimal. UVA and UVB radiation, on the other hand, are not absorbed by ozone and are readily absorbed by the skin. It follows that UVA and UVB radiation are the most dangerous types of UV radiation to human skin. UVB penetrates the skin superficially and is the principle cause of sunburn. UVA radiation, on the other hand, penetrates the dermis of the skin more deeply, causing a slow natural tan to develop, while also contributing to burning. UVB and UVA are both believed to play a significant role in the development of various types of skin cancers. Both of these types of radiation can generate reactive oxygen species (i.e., “free radicals”) upon contacting the skin. These reactive species can alter the structure of RNA and DNA of skin cells, eventually causing cancer.

To counter the adverse affects of UV radiation on the skin, topical sunscreen lotions, creams, sprays, and the like have been developed. These sunscreens typically contain chemicals designed to protect the skin by absorbing, reflecting, or scattering UV radiation. Cosmetic companies and dermatologists claim that these sunscreens are effective to block UVA and UVB radiation and thus reduce one's probability of contracting skin cancer. In fact, the American Academy of Dermatology, the American Academy of Pediatrics, and the Skin Cancer Foundation recommend that sunscreens with substantial SPF factors be used from an early age to prevent skin damage.

Nevertheless, recent studies have shown that many sunscreen products may actually increase the incidence of cancer, rather than reducing it. In particular, the chemicals and oxygen content in many sunscreen products may cause cancer by increasing the generation of free radicals on and within the skin. These free radicals may react with molecules in the skin to damage the fat, DNA, RNA, and proteins of skin cells. This damage causes the skin to age and is believed to be a principal cause of skin cancer.

In view of the foregoing, what are needed are products and methods to reduce the effects of free radicals on or within the skin. Further needed are products and methods to negate or counteract the damaging effects of many sunscreen products.

### SUMMARY

The invention has been developed in response to the present state of the art and, in particular, in response to the problems and needs in the art that have not yet been fully solved by currently available methods and products. Accordingly, the invention has been developed to provide products and methods for reducing the effects of free radicals on, within, or under the skin. The features and advantages of the invention will become more fully apparent from the following description and appended claims, or may be learned by practice of the invention as set forth hereinafter.

Consistent with the foregoing, a topical product to protect the skin from free radicals is disclosed in one embodiment of the invention as including a carrier medium for topical application to the skin. A sacrificial material which is compatible with the skin is interspersed with the carrier medium. This sacrificial material may be selected to react with free radicals within the skin. The sacrificial material may be further selected to react with water to produce hydrogen (a powerful antioxidant) and a reaction product that is also compatible with the skin. In this way, both the sacrificial material and the hydrogen may neutralize free radicals.

In selected embodiments, the sacrificial material includes an elemental metal and/or a metal alloy. For example, the sacrificial material may include one or more of Fe, Mg, Al, Zn, Cu, Ca, C, Bi, Ti, Ta, In, Ni, Nb, Pd, or the like. In certain embodiments, the elemental metal or metal alloy may be provided in powder form. The metal powder may be in wet or dry form and may be suspended particles or colloids in a liquid, paste, gel, or the like. In certain embodiments, the mean particle size of the powder may be selected to control the rate at which the sacrificial material reacts with the water. In certain embodiments, the topical product includes water. The water may be mixed with the sacrificial material prior to applying the topical product to the skin or, alternatively, at the time the topical product is applied to the skin. In other embodiments, the topical product may use the water that is contained within the skin.

In yet another embodiment, a topical product for protecting the skin from free radicals may include a carrier medium for topical application to the skin. A sacrificial material comprising skin-compatible bacteria may be interspersed with the carrier medium. These skin-compatible bacteria may infuse the skin with sacrificial cells that may shield the skin from the effects of free radicals. In selected embodiments, the sacrificial material is yogurt. Accordingly, the skin-compatible bacteria may include one or more of lactobacillus acidophilus, lactobacillus casei, and bifidobacterium, each of which may be found in yogurt.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 shows the possible effects of a topical product that contains zinc metal as the sacrificial material;

FIG. 2 shows the possible effects of a topical product that contains magnesium metal as the sacrificial material;

FIG. 3 shows the possible effects of a topical product that contains iron metal as the sacrificial material;

FIG. 4 shows one embodiment of a tube that may contain a topical product in accordance with the invention;

FIG. 5 shows another embodiment of a tube that may contain a topical product in accordance with the invention; and

FIG. 6 shown one embodiment of a method for using a topical product in accordance with the invention.

### DETAILED DESCRIPTION OF THE INVENTION

It will be readily understood that the components of the present invention, as generally described and illustrated herein, could be arranged and designed in a wide variety of different configurations. Thus, the following more detailed description of the embodiments of the invention is not intended to limit the scope of the invention, but is merely representative of certain examples of presently contemplated embodiments in accordance with the invention.

A sacrificial material, as referred to herein, refers to a metal or other material used to protect another material from oxidation, corrosion, or damage. More generally, a sacrificial material may be defined as a material that is more easily oxidized, or corroded, than a material that it protects. For example, when zinc and iron metals are combined together in the presence of oxygen, the zinc and iron may both lose electrons. Because iron is less reactive than zinc, however, it may tend to replace its lost electrons with electrons from the zinc. As a result, the iron will tend to act as a neutral atom and the zinc will react with the oxygen. The iron will not corrode until all of the zinc is oxidized. Sacrificial materials (e.g., sacrificial anodes) are used in cathodic protection systems to protect metal structures such as hulls of ships, steel pipelines, copper tanks of water heaters, among many other structures. Materials used in sacrificial anodes may include zinc, aluminum, magnesium, or the like.

In selected embodiments in accordance with the invention, a cream, lotion, spray, liquid, ointment, gel, or the like (hereinafter referred to as a “topical product”) may be infused with a sacrificial material. The topical product may be applied to the skin to protect the skin from the harmful effects of sunlight, and more particularly form free radicals such as superoxide, hydrogen peroxide, and hydroxyl radicals. More specifically, the sacrificial material may be designed to react with free radicals generated by ultraviolet radiation, and more particularly UVA and UVB radiation, when it contacts the skin. Ideally, the reaction between the sacrificial material and the free radicals will generate a reaction product that is compatible with (and potentially beneficial to) the skin. Like the sacrificial anodes used in conventional applications (i.e, bridges, hot water heaters, etc.), the sacrificial material in the topical product may react with an oxidizing agent (in this example free radicals) to prevent the free radicals from reacting with, and thereby damaging, a host structure (in this example skin cells).

In selected embodiments, the sacrificial material may include various elements or materials that are compatible with (i.e., harmless and/or non-irritating to) the skin, while also producing a reaction product that is compatible with the skin. Such materials may include, for example, Fe, Mg, Al, Zn, Cu, Ca, C, Bi, Ti, Ta, In, Ni, Nb, Pd, Ni—La alloys, various metal hydrides, hydrogen gas, or any other material that readily reacts with free radicals. Hydrogen gas may be particularly desirable because it is such a small molecule. As a result, hydrogen may achieve more intimate contact with free radicals and thus be particularly effective at taking up and neutralizing free radicals. Thus, in selected embodiments, materials that generate or store hydrogen may be particularly desirable. In selected embodiments, the sacrificial material may include skin-compatible materials that oxidize (i.e., react with free radicals) at temperatures between about 5° C. and 50° C.

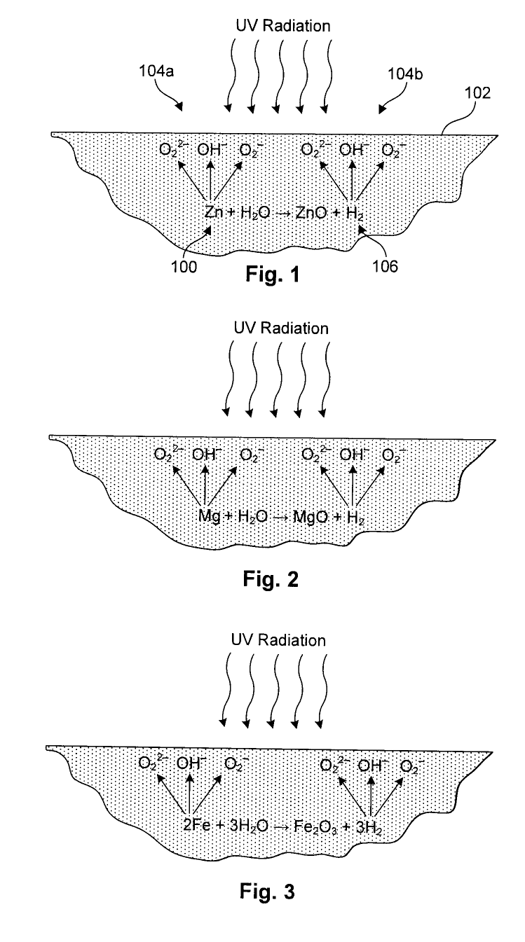

In certain embodiments in accordance with the invention, the sacrificial material may include a hydrogen-generating material. For example, referring to FIG. 1, zinc metal 100, when applied to the skin 102, may react with free radicals 104a to produce zinc oxide or other zinc compounds. Zinc metal 100 may also react with water to form zinc oxide and hydrogen gas 106. As mentioned above, hydrogen 106 may provide a sacrificial material that is particularly effective at reacting with free radicals 104b. Thus, the sacrificial material, in this example zinc 100, may itself react with free radicals 104a, and may generate hydrogen 106 which may also react with free radicals 104b, effectively providing two different materials for neutralizing free radicals 104a, 104b. FIGS. 2 and 3 show two other metals (Mg and Fe) that will react with free radicals, while also reacting with water to generate hydrogen to further react with free radicals. This double action provides a more powerful way to neutralize free radicals than typical antioxidants.

In other embodiments, the sacrificial material may simply include an oxygen-reacting material, such as a material listed above, which may readily react with oxygen free radicals. In yet other embodiments, the sacrificial anode material may include a hydrogen-absorbent compound, such as various metal hydrides, C, Pd, Ni—La alloys, or the like. This hydrogen-absorbent compound may be used to store hydrogen and release the hydrogen upon applying the topical product to the skin, upon applying ultraviolet radiation to the compound, or upon placing the compound in intimate contact with reactive oxygen species such as free radicals. For example, various metal hydrides (e.g., nickel hydride) are known to both store and discharge significant amounts of hydrogen at room temperature. Other metal hydrides may discharge hydrogen when warmed, such as when applied to skin or warmed by sunlight. In yet other embodiments, the sacrificial material may include a hydrogen-saturated material.

In selected embodiments, a sacrificial material may include various skin-compatible materials that release hydrogen. This hydrogen may react with free radicals to form water and skin-compatible reaction byproducts. For example, the sacrificial material may include skin-compatible carbons or skin-compatible activated charcoal which can react with oxygen or hydroxyl ions or radicals to form carbon dioxide and water. In yet other embodiments, the sacrificial material may include skin- or human-compatible organisms such as bacteria, enzymes, viruses, or the like. These organisms may defend the skin against free radicals by providing sacrificial cells to protect healthy skin cells, DNA, or the like. In other words, these sacrificial cells may be infused into the skin to incur damage that would otherwise happen to healthy skin cells or DNA. Such organisms may include or be present in yogurt, yeast, certain types of e. coli that are not harmful to humans, or the like. Yogurt in particular may include skin-compatible bacteria such as lactobacillus acidophilus, lactobacillus casei, and/or bifidobacterium.

In selected embodiments, the sacrificial material may be incorporated into a topical product, such as a cream, lotion, spray, liquid, ointment, gel, or the like, that is not a sunscreen product. Thus, the topical product may not block UV radiation but may actually allow the skin to burn. This may provide a warning to a user that he or she should remove him or herself from the sunlight. One advantage of such a product is that it may allow ultraviolet rays to penetrate the skin and thereby aid in vitamin D production. It has been shown that use of a sunscreen product with a sun protection factor (SPF) of 8 inhibits greater than 95 percent of the body's production of vitamin D. Vitamin D is a powerful antioxidant the lack of which may contribute to cancer. A generally accepted guideline is that 15 minutes of sunlight per day will produce an optimal amount of vitamin D in the body.

In other embodiments, however, the sacrificial material may be incorporated into a topical product such as a sunscreen product having an SPF factor. Such a product may be applied to the skin to avoid or delay sunburns while providing added protection against free radicals generated in the skin. Such a product may also be used to counteract the additional free radicals that may be produced by the sunscreen itself. That is, the sunscreen may contain oxygen or oxygen-containing compounds that may produce free radicals in the presence of ultraviolet radiation, in addition to the free radicals that are naturally produced by the incidence of sunlight on the skin. Thus, the sacrificial material may be used to react with, and thus counteract the effect of, the additional free radicals created by the sunscreen itself.

In selected embodiments, the topical product may be designed to generate localized heat on the skin in the presence of UV radiation. For example, various metals such as zinc, magnesium, iron, or the like may generate heat when they react with oxygen free radicals. In selected embodiments, this heat may be used advantageously to notify a user of the product that he or she is in the presence of ultraviolet radiation. The amount of heat generated may depend on the choice of sacrificial material, as well as other factors such as the concentration or particle size of the sacrificial material. The amount of heat generated may also depend on the amount of exposure to UV radiation.

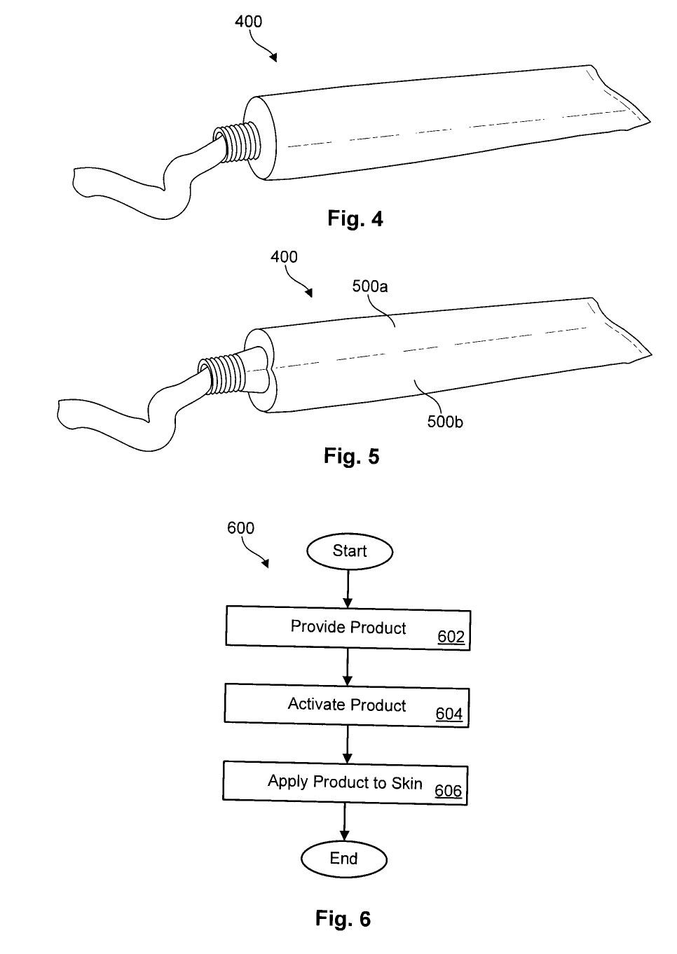

Referring to FIG. 4, in selected embodiments, a topical product 400 in accordance with the invention may include a cream, lotion, ointment, or other topical preparation for application to the skin. In selected embodiments, the topical product 400 may contain any of the sacrificial materials described above suspended in a vehicle such as a cream or other topical preparation. When a user wishes to utilize the topical product 400, the user may combine the topical product 400 with a small amount of water in the hand or other container and apply the product 400 to the skin. In other embodiments, the water may be contained within the topical product itself. In other embodiments, the topical product 400 may use the moisture in the skin itself. In yet other embodiments (as shown in FIG. 5), the topical product 400 may include two parts: a first part 500a that contains the sacrificial material and a second part 500b that contains an aqueous-based cream. The contents of either part 500a, 500b may be embodied as a cream, lotion, or ointment. For example, the first part 500a may comprise magnesium or zinc as suspended colloids in an oil and the second part 500b may be a cream that has a high water content. The contents of each part 500a, 500b may be mixed upon dispensing the topical product 400 and/or applying it to the skin. In the illustrated embodiment, a two-part tube is used to contain each part 500a, 500b.

Upon applying the topical product 400 to the skin, the water may react with any metal powders contained therein to generate heat and hydrogen for neutralizing free radicals on or within the skin. The heat may improve the permeability of the skin and help the skin absorb the topical product. The metal powder and water may generate reaction products (e.g., zinc oxide, magnesium oxide, aluminum oxide, iron oxide, calcium hydroxide, etc.) that are compatible with and potentially beneficial to the skin.

Referring to FIG. 6, in certain embodiments, a method 600 for using a topical product in accordance with the invention may include initially providing 602 a topical product and optionally activating 604 the product. Activating 604 may include initiating the heat- and/or hydrogen-producing chemical reaction by combining the product with water. Before, during, or after the product is activated 604, the product may be applied 606 to the skin. In certain embodiments, applying 606 may include rubbing a topical product such as a cream or ointment into the skin.

It should be recognized that the methods and apparatus discussed herein may be applied to a wide variety of products, each of which is intended to be captured within the scope of the present invention. For example, the apparatus and methods discussed herein may be applied to products such as facial masks, body wraps, mud masks, sprays, bathing products, massage products, body powders, moisturizers, bandages, or the like.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. A topical product to neutralize free radicals in the skin, the topical product comprising:
a carrier medium for topical application to the skin;
a sacrificial material interspersed with the carrier medium and compatible with the skin, the sacrificial material selected to react with free radicals within the skin; and
the sacrificial material further configured to react with water to produce hydrogen and a reaction product that is also compatible with the skin.

2. The topical product of claim 1, wherein the sacrificial material comprises an elemental metal.

3. The topical product of claim 2, wherein the elemental metal comprises a metal selected from Fe, Mg, Al, Zn, Cu, Ca, C, Bi, Ti, Ta, In, Ni, Nb, Pd and combinations thereof.

4. The topical product of claim 2, wherein the elemental metal comprises a metal powder.

5. The topical product of claim 4, wherein the mean particle size of the metal powder is selected to control the rate at which the sacrificial material reacts with the water.

6. The topical product of claim 1, wherein the sacrificial material comprises a metal alloy.

7. The topical product of claim 1, wherein the topical product comprises water.

8. The topical product of claim 7, wherein the water is mixed with the sacrificial material prior to application to the skin.

9. The topical product of claim 7, wherein the water is mixed with the sacrificial material at the time of application to the skin.

10. A method for protecting the skin from free radicals, the method comprising:
applying a carrier medium infused with a sacrificial material to the skin, the sacrificial material compatible with the skin, the sacrificial material selected to react with free radicals within the skin; and
reacting the sacrificial material with water to produce hydrogen and a reaction product that is also compatible with the skin.

11. The method of claim 10, wherein the sacrificial material comprises an elemental metal.

12. The method of claim 11, wherein the elemental metal comprises a metal chosen from Fe, Mg, Al, Zn, Cu, Ca, C, Bi, Ti, Ta, In, Ni, Nb, Pd and combinations thereof.

13. The method of claim 11, wherein the elemental metal comprises a metal powder.

14. The method of claim 13, wherein the mean particle size of the metal powder is selected to control the rate at which the sacrificial material reacts with the water.

15. The method of claim 10, wherein the sacrificial material comprises a metal alloy.

16. The method of claim 10, further comprising mixing the topical product with water prior to applying the topical product to the skin.

17. The method of claim 10, further comprising mixing the topical product with water at the time of applying the topical product to the skin.

18. A topical product to protect the skin from free radicals, the topical product comprising:
a carrier medium for topical application to the skin; and
a sacrificial material interspersed with the carrier medium and comprising skin-compatible organism, the skin-compatible organism neutralizing the effects of free radicals within the skin.

19. The topical product of claim 18, wherein the sacrificial material is yogurt.

20. The topical product of claim 18, wherein the skin-compatible organism comprises bacteria selected from lactobacillus acidophilus, lactobacillus casei, bifidobacterium and combinations thereof.

21. The topical product of claim 18, wherein the skin-compatible organism comprises e. coli.

22. The topical product of claim 18, wherein the skin-compatible organism comprises yeast.
